SEQUENCE LISTING (6 sequences)

SEQ ID NO: 1 - DNA, 69 bp, Artificial Sequence
  synthetic construct
cggccgtgcgtacttagacatgcatggcttaatctttgagacaagcatatgctactggcaggatcaacc

SEQ ID NO: 2 - DNA, 60 bp, Artificial Sequence
  synthetic construct
aagccagctgcttccattggtgggtcgtttttgctgtcaccagcaacattgcctcgtcta

SEQ ID NO: 3 - DNA, 60 bp, Artificial Sequence
  synthetic construct
gcgttactcacccgtccgccactcgtcagcatcctaagacctgttaccgttcgacttgca

SEQ ID NO: 4 - DNA, 20 bp, Artificial Sequence
  synthetic construct
tgcaagtcgaacggtaacag

SEQ ID NO: 5 - DNA, 17 bp, Artificial Sequence
  synthetic construct
gcgttactcacccgtcc

SEQ ID NO: 6 - DNA, 23 bp, Artificial Sequence
  synthetic construct
cgccactcgtcagcatcctaaga

(19) United States
(12) Patent Application Publication
(10) Pub. No.: US 2013/0231261 A1
(43) Pub. Date: Sep. 5, 2013

(54) RNASE H-BASED RNA PROFILING

(71) Applicant: INSTITUTE FOR SYSTEMS BIOLOGY, Seattle, WA (US)

(72) Inventors: Adrian OZINSKY, Seattle, WA (US); Rolf KUESTNER, Bothell, WA (US); Gregory ZORNETZER, Seattle, WA (US)

(73) Assignee: INSTITUTE FOR SYSTEMS BIOLOGY, Seattle, WA (US)

(21) Appl. No.: 13/769,195

(22) Filed: Feb. 15, 2013

Related U.S. Application Data
(60) Provisional application No. 61/600,486, filed on Feb. 17, 2012.

(51) Int. Cl. C12Q 1/68

(52) U.S. Cl. CPC C12Q 1/6823
USPC 506/9; 435/6.11

(57) ABSTRACT

Methods for determining the presence, absence and/or amount and/or identity of RNA in a biological or other sample employs capturing and separating a DNA:RNA hybrid formed by a DNA probe and the RNA of interest from unhybridized DNA and RNA with an RNase H under conditions wherein the nuclease activity of the RNase H is inhibited, releasing the DNA probe by altering the conditions so that the nuclease activity is restored, and determining the presence or amount, and, for multiplex samples, nature of the DNA released. The method can be used to determine RNA of various types, including RNA comprising transcriptomes.

### RELATED APPLICATION

This application claims benefit of U.S. provisional application Ser. No. 61/600,486 filed 17 Feb. 2012 which is incorporated herein by reference in its entirety.

### TECHNICAL FIELD

The invention relates to analysis of samples for the presence or amount of RNA species of interest. The method may be multiplexed so that multiple species of RNA can be so detected and quantified by virtue of the characteristics of a diagnostic DNA oligomer probe hybridizing to said RNA and captured and released by incubation with RNase H under suitable conditions.

### BACKGROUND ART

Determination of the nature, presence, or amount of RNA molecules in biological samples is typically done by a variety of methods, including preparing cDNA which can then be probed and detected, or by probing, for example, Northern blots using probes of known sequence. Such methods are difficult to multiplex and to reduce in size. The invention described below addresses these problems.

The use of RNase H to isolate DNA:RNA hybrids has been contemplated in U.S. Pat. No. 7,560,232. In the methods described, the detection of the amount of complex formed is the endpoint rather than utilizing the capture DNA as the criterion for assay of RNA in a sample. It is suggested to use an RNase H that lacks nuclease activity and various mutant RNase H alternatives are disclosed. It is also pointed out that the nuclease activity is present only in the presence of magnesium ion, but alteration of nuclease activity by the presence or absence of magnesium ion is never employed to release and expose for further analysis the DNA oligomer that forms a member of the hybrid.

The present invention takes advantage of the reversible nuclease activity of RNase H due to the presence or absence of magnesium ion to utilize DNA probes in a prepared oligomer library as indices for the presence and/or amount of RNA complementary to them.

### DISCLOSURE OF THE INVENTION

The invention provides a method that is easy to multiplex and to miniaturize for detecting, quantitating, and identifying any RNA species in a sample. The method can be applied to detection and quantitation of a single RNA species, or to a multiplicity of RNA species in a sample or to a multiplicity of samples. If a multiplicity of RNA species is assayed, methods to distinguish various DNA oligomers used to hybridize to these species may be included using methods standard to the art. In particular, Next-Generation Sequencing methods, as described in Shendure and Ji, Nature Biotechnology (2008) 26:1135-1145, are well-suited to the application of quantifying different sequences.

The invention provides a method that is easy to multiplex so as to detect and quantify the RNA species of different samples, simultaneously. After hybridizing the RNA of each sample to a distinct set of DNA oligomers, the samples can be pooled, and after RNase H-based hybrid purification, analysis of the DNA will provide a metric for the identity and the abundance of the RNA content of each sample. In this case, methods to distinguish various sets of DNA oligomers used to hybridize to these different samples should be included, as noted above.

Thus, in one aspect, the invention is directed to a method to detect and/or quantify, and/or identify at least one RNA of interest in a sample, comprising the steps of:

a) exposing the sample to a DNA probe comprising a sequence complementary to said at least one RNA of interest to form DNA:RNA hybrids with any said RNA in the sample;

b) incubating the DNA:RNA hybrids formed in a) with RNase H under conditions that inhibit the nuclease activity of the RNase H but do not inhibit its DNA:RNA binding activity;

c) separating RNase H-bound DNA:RNA from unbound DNA and RNA;

d) incubating the RNase H-bound DNA:RNA under conditions wherein the nuclease activity is restored so as to hydrolyze the RNA of the DNA:RNA hybrids and release the DNA probe; and

e) determining the presence and/or amount and/or identity of the released DNA probe, thereby determining the presence and/or quantity and/or identity of the at least one RNA of interest in the sample.

As noted above, the assay can be multiplexed to determine two or more, or large numbers of, RNA species in a single sample and the method is similar to that with respect to the single species except that some means to distinguish the various DNA complements of the RNA species is provided. This can be done simply by sequencing the liberated DNA oligomers, or by using primers/probes and PCR (or real-time PCR) to distinguish and quantify the individual DNAs, or the DNA oligomeric probes may be identified by hybridization to a microarray, or be differentially labeled. It is possible to generate multihued particulate labels of nanoparticle size as described in U.S. Pat. Nos. 6,642,062 and 6,492,125 to provide a large number of different labels so that a multiplicity, i.e., 2 or more DNA probes can be determined after separation of individual nanoparticles.

The labels themselves may be used for quantitation if they are detectable. For example, if fluorescent labels are used, the intensity of fluorescence may be determined as a measure of quantity or concentration. Radioactive labels could also be used where, again, the level of radiation is an index of quantity. Various methods of labeling the DNA probes in a multiplexed library are available in the art.

The method may also be miniaturized by conducting all or parts of the invention method in a microfluidic system supplying the various reagents in nanoliter or picoliter quantities, thus permitting assays using limited quantities of RNA.

### BRIEF DESCRIPTION OF THE DRAWINGS

FIG. 1 shows a diagram of the principle on which the method is based, including the main steps of the method. R1 represents an RNA molecule within the sample to be quantified and RX represents all RNA molecules within the sample that are not quantified. D1 represents the DNA oligomer used to probe the RNA sample. The circle with RNase H inscribed represents RNase H attached to a solid support. The diagram depicts profiling of only one RNA type of RNA molecule, but the method is general to a mixture of many RNA molecules and complementary DNA oligomers, where R1 would hybridize to D1, R2 to D2, R3 to D3, etc.

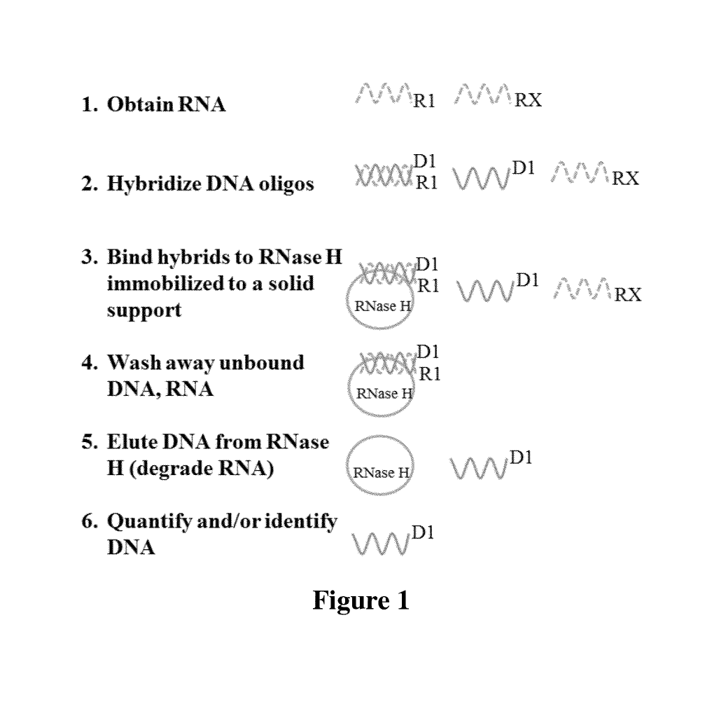

FIG. 2 shows a comparison of results of the current invention method using RNase H versus cDNA-based analysis of intact RNA and formalin fixed paraffin embedded (FFPE) RNA.

### MODES OF CARRYING OUT THE INVENTION

The method of the invention may be used to determine the presence and/or amount and/or identity of one or a multiplicity of RNA species of interest in a sample. The multiplicity may include the 2 or more RNA species, 3 or more or 10 or 100 or more or 1,000 or more or 10,000 or more. Each specific integer in these intervals is to be considered as specifically disclosed. Any type of RNA is suitable to the method, including microRNA and its variants, mRNA, tRNA, ribosomal RNA, non-coding RNA and the like. Since it is complementary sequences in the DNA probes that will be analyzed, the length of the target RNA to be determined does not matter as long as it contains a sufficiently distinctive portion to hybridize uniquely to an oligomeric DNA.

For example, “microRNA” (or miRNA) refers to any type of interfering RNA, including endogenous microRNA and artificial microRNA. Endogenous microRNA are small RNAs naturally present in the genome which are capable of modulating the productive utilization of mRNA; also included are RNA sequences, other than endogenous microRNA, that are capable of modulating the productive utilization of mRNA.

Also subject to the invention method are noncoding RNA gene products with important functional roles in regulation of gene expression, developmental timing, viral surveillance, immunity, inflammation and oncogenesis. Not only the classic transfer RNAs (tRNAs) and ribosomal RNAs (rRNAs), but also small nuclear RNAs (snRNAs), small nucleolar RNAs (snoRNAs), small interfering RNAs (siRNAs), tiny non-coding RNAs (tncRNAs), repeat-associated small interfering RNAs (rasiRNAs) and microRNAs (miRNAs) are now believed to act in diverse cellular processes such as chromosome maintenance, gene imprinting, pre-mRNA splicing, guiding RNA modifications, transcriptional regulation, and the control of mRNA translation. Target RNA molecules can also include variant sequences including nucleotide substitutions, deletions, insertions. DNA polymorphisms can be the source of variant sequences.

The sample in which the RNA is to be detected may be a biological sample, but other types of samples may also be subject to the method of the invention. Biological samples include extracts from tissues, bodily fluids such as serum, plasma, urine, semen, cerebral spinal fluid, and the like as well as saliva. Extracts of plant tissue or microbial sources may also be used. The sample may be subjected to suitable pretreatment prior to carrying out the method of the invention. Other samples might include those prepared synthetically to contain RNA, such as those to be used as reagents or control samples, as well as pharmaceutical or veterinary compositions, such as those that contain interfering RNAs. The nature of the sample will depend on the interest of the practitioner.

One particularly advantageous application for the method of the invention relates to the analysis of any sample where the RNA might be degraded or fragmented by time, storage condition, or composition of the entire sample. This would include tissue samples that have been preserved, often by formalin fixation/paraffin embedding (FFPE), a method that preserves the physical architecture and the protein component of the tissue but causes damage to RNA. It has long been considered desirable to perform RNA analysis on samples derived from FFPE-treated tissues, but the degradation and damage to the RNA presents difficulties. The present method minimizes the effect of the damage to the RNA on the validity of the assay. Other examples include prehistoric/historic samples/repository samples, sub-optimally processed or stored samples, and samples that might include high concentrations of nucleases.

The length of sequence in the DNA oligomer needed to characterize a particular RNA is dependent on the nature of the RNA; in general a length of 10 nucleotides is considered the minimum based on the binding requirements of RNase H, but longer sequences are generally necessary to ensure specificity. For microRNA's the common sizes for hybridization to the DNA oligonucleotide will be 18-25 nucleotides, though pre-processed forms also might be appropriately detected and that are longer in length. For mRNA the common sizes for hybridization generally range from 25-60 nucleotides, but could be longer. In any case, a library of oligomeric DNA's is prepared according to the sequences of RNA of interest to be determined. (In the case of determining only a single species, this would be a library of one.) Thus, the library will contain probes that contain complementary sequences of sufficient length to bind specifically to the RNA species in the sample that are of interest. A desired length of this sequence can be accommodated by the RNase H since the binding site involves 9-10 nucleotides and simultaneous binding of more than one RNase H molecule to the probe can enhance the avidity in the case of longer sequences. This will increase the effectiveness of the capture of RNA species and enable the capture of low abundance or very dilute RNA species. For example, simultaneous binding of three RNase H molecules to a 36 nucleotide DNA:RNA hybrid has been demonstrated.

If only one RNA is to be detected or quantified, means for identifying the specific DNA oligomer to which it is bound are optional; if the system is multiplexed to detect or quantify many different RNA species in the same sample, or to detect a set of RNA species in a collection of samples that are optionally handled in a pooled manner after DNA:RNA hybridization, each DNA oligomer probe should contain means of identifying which probe is being quantitated as a measure of its target RNA.

The oligomeric probes, as noted above, must contain sequences of sufficient length to hybridize specifically to their complementary target RNAs. The probes may contain additional sequences besides the region of complementarity, however. These “extensions” of the complementary portion are useful as labels to distinguish various different DNA oligomer probes targeted to different RNAs, or to the same RNAs present in a collection of samples that have been handled in a pooled manner after DNA:RNA hybridization. For example, the extension may contain a nucleotide bar code—i.e., a sequence of nucleotides that specifically characterizes the oligomer, or that specifically characterizes a particular library of DNA oligomers that is being used for a particular sample, and is distinct from another library of DNA oligomers that is being used to hybridize to the RNA targets in a separate sample. This sequence will differ depending on the RNA to which the DNA probe is targeted or will differ based on the number of different samples that are being processed in combination. The extensions may also contain binding reagents or labels thus enabling detectable labeling of the oligomers. The extensions may also contain primer sequences that permit amplification of the DNA probes or portions thereof when they are recovered, or permit hybridization of the DNA oligomers to complementary sequences for capture, detection or quantification. The primer sequences may be the same for all of the oligomers in a multiplex system if alternative means for identification or separation are provided or may be themselves the means for distinguishing the various probes used in the multiplex. The arrangement of these features of the extended oligomers is variable and subject to conventional design considerations. The design and placement of features in such extensions is within ordinary skill and there are many variations possible.

Since any of the foregoing features may serve to label and identify a particular DNA probe, these features, such as bar codes, primer sequences and the like are collectively referred to as “labels.” Detectable labels are those whose level can be conveniently be measured quantitatively, such as fluorescent labels, radioactive isotopes, chromophores and the like.

Methods for synthesis of appropriate DNA oligomers are well within ordinary skill. A DNA library of oligomers needs to include complements of the RNA species of interest or the relevant portions thereof. If the RNA species in the sample are to be quantified, an excess of the complementary DNA oligomer should be used so that the oligomer is not a limiting reagent.

If desired, a preliminary step to eliminate RNA species known to be present, but not of interest in the sample, may be performed to simplify the method of the invention as applied to the desired targets. In one approach, a set of “subtraction” DNA oligomers is employed to hybridize to these unwanted RNA species. The resulting hybrids can then be removed from the sample using the techniques of the present invention—i.e., nuclease-inactivated RNase H, or by using any specific binding agent for such hybrids, such as antibodies. This can be particularly advantageous if the RNA species of interest are present only in relatively small amounts. (“Antibodies” as used herein includes complete antibodies as well as simply the immunospecific portions thereof, including recombinantly produced single-chain antibodies.)

A method for reducing noise caused by the nonspecific adhesion of species other than RNA:DNA hybrids is the addition of enzymatic treatment with a single-stranded specific nuclease, such as Micrococcal S7 nuclease or Aspergillus nuclease S1, that hydrolyzes DNA and RNA that is not part of a duplex. This would reduce the concentration of unhybridized DNA oligomers as well as the concentration of unbound RNA in the sample. A nuclease specific for single-stranded RNA, including RNase A and RNase T1 may also be used to digest RNA molecules not participating in RNA:DNA hybrids. Alternatively, the sample can be treated with a protein that preferentially binds to single-stranded nucleic acids, thus removing them from the pool that will bind the RNase H. These noise reduction steps may be performed during or between steps a), b) and c).

Another method for reducing noise caused by the nonspecific adhesion of species other than RNA:DNA hybrids is to use capture oligonucleotide DNA probes that hybridize to the RNA at closely adjacent positions. These DNA oligonucleotides then can be ligated together by addition of a DNA ligase, such as T4 DNA ligase, whose activity requires a double stranded substrate and has very low activity to ligate single stranded oligonucleotides. The detection method (sequencing, PCR, etc.) then would detect the ligated form of the capture oligonucleotide, and distinguish it from the unligated form, reducing noise.

Unless otherwise noted or apparent from context, “a” or “an” means “one or at least one” or “one or more than one.”

Components of the Method

RNase H refers to any protein or protein derivative capable of specifically binding a duplex of DNA:RNA and hydrolyzing the RNA component of the duplex to produce 5′-phospho mononucleotides. This behavior is described as EC 3.1.26.4 by the Nomenclature Committee of the International Union of Biochemistry and Molecular Biology (IUBMB). In particular, these variants generally fall into three broad categories: termed RNase H1, RNase H2, and RNase H3 based on sequence analysis. Examples of RNase H1 and RNase H2 are the E. coli genes rnhA and rnhB, respectively. This function is required for life, and many variants of this protein are known in the art, including ones with stability and activity extremes of temperature, salinity and other conditions.

Various forms of RNase H may be used according to the ambient conditions of the methods. For example, RNase H's which are sufficiently thermostable to temperatures of more than 50° C. are available, as well as cold-adapted forms that are operative between 1° C. and 4° C. These forms may be used in steps b), c) and d).

It is convenient for the RNase H to be supplied in immobilized form for ease of separation. The RNase H may be immobilized on a column or on beads using materials generally known in the art. In one approach, the RNase H may be coupled to an affinity tag for binding to the solid support. One example is coupling of the RNase H to biotin or streptavidin binding peptide (SBP). See Keefe, et al., Protein Expression and Purification, (2001) 23:440-446 then immobilized on a streptavidin coupled solid support. Alternatively, the RNase H is coupled with a small hapten (e.g., digoxin) and the solid support is conjugated with an anti-hapten polypeptide variant (e.g., anti-digoxin antibody) or vice versa. Using this approach, immobilization of the RNase H may be done before, during or after incubation with the sample containing DNA:RNA hybrids.

Solid supports for immobilizing the RNase H to include materials such as acrylamide, cellulose, nitrocellulose, glass, polystyrene, polyethylene vinyl acetate, polypropylene, polymethacrylate, polyethylene, polyethylene oxide, polysilicates, polycarbonates, Teflon®, fluorocarbons, nylon, silicon rubber, polyanhydrides, polyglycolic acid, polylactic acid, polyorthoesters, polypropylfumerate, collagen, glycosaminoglycans, and polyamino acids. The solid substrate may be in the form of thin films or membranes, beads, bottles, columns, dishes, fibers, tubes, slides, woven fibers, shaped polymers, particles and microparticles. Magnetic beads may be especially advantageous.

The RNase H may also be directly conjugated to the solid substrate via reactive groups, wherein the material comprising the solid support has reactive groups such as carboxy, amino, hydroxy, etc., which are used for covalent or non-covalent attachment of the RNase H. Conjugation to the solid substrate may also be through one or more intervening components.

The use of RNase H as a DNA:RNA specific protein may, in one embodiment, be used in a modification of the method described in PCT publication WO2011/097528 incorporated herein by reference. WO2011/097528 describes assays for RNA wherein hybrids are formed between the target RNA and DNA oligomers which are then isolated. Antibodies to the hybrids are illustrated as one method of isolation. In the method illustrated, the bound DNA was released by denaturing the hybrid and then sequenced using commercially available sequencing techniques employing signature (primer host) sequences for binding to primers for amplification and suitable bar codes. In the method of the present invention, the hybrids (or host primer) may be isolated using RNase H rather than the antibody illustrated and the DNA probe released by the restored nuclease activity. It can then be directly quantitated and/or sequenced.

So long as their relevant function is maintained, DNA probes or subtraction DNA oligomers may include modified forms. It should be emphasized, of course, that only those modifications that do not disturb the ability of the hybrid to form or the ability of the hybrid to couple to RNase H may be included in large quantities. Indeed, these modifications may be more appropriate on the above described extensions of the portions of the DNA probes designed to bind the target RNA, where interference with hybridization or binding is less an issue.

Modifications to the base moiety include natural and synthetic modifications of A, C, G, and T/U as well as alternate purine or pyrimidine bases.

Other modified forms include those where additional groups are covalently attached, modifications to sugars and modifications to backbone linkages.

The DNA probes thus may also comprise locked nucleic acid (LNA™) monomers in which the ribose ring is locked into the ideal conformation for base stacking and backbone pre-organization and can be used like a regular nucleotide. The nucleic acid contains a methylene bridge connecting the 2′-O and the 4′-C. The locked structure increases the stability of oligonucleotides by increasing the melting temperature. Two forms of locked nucleic acids are possible: first, the β-D ribo variety, commercially available as LNA™, second the α-L variety. Both forms increase the stability of a nucleic acid double helix.

Modifications to the sugar moiety include natural modifications of the ribose and deoxyribose as well as synthetic modifications and sugar analogs (including the locked nucleic acids mentioned above). In addition, the literature has described cyclohexene nucleic acids, which replace the ribose ring with a cyclohexene ring and nucleic acids based on an arabinose rather than ribose/deoxyribose ring (arabinonucleic acids).

Modified forms may also be modified at the phosphate moiety, including but not limited to, those resulting in a phosphorothioate, chiral phosphorothioate, phosphorodithioate, phosphotriester, aminoalkylphosphotriester, methyl and other alkyl phosphonates including 3′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, and boranophosphate linkages. It is understood that these phosphate or modified phosphate linkages can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

Other alternatives to phosphodiester linkages include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

As noted above, additional moieties may be covalently attached to the DNA, including fluorescent labels (such as 5-Carboxyfluorescein), affinity tags (such as biotin), and molecules affecting stability (such as a DNA minor groove binder).

The DNA probes may contain only a single modification, or multiple modifications within one of the moieties or between different moieties. For example, the DNA probes may have both the sugar and the phosphate moieties of the nucleotides replaced, by for example an amide type linkage (aminoethylglycine) (PNA). See, e.g., U.S. Pat. Nos. 5,539,082; 5,714,331; and 5,719,262.

In summary, “DNA” for use as a probe in the methods as defined herein includes modified forms of DNA as described above.

The foregoing components can be packaged for convenience or commercialization in the form of a kit with RNase H and/or appropriate DNA probes packaged in appropriate containers and with additional reagents if desired, such as buffers useful in performing the method, along with instructions for its conduct. Thus, the invention includes such kits.

Methods of Inactivating and Reactivating RNase H

Various methods may be used to reversibly inactivate the catalytic activity of RNase H (degradation of RNA) while not affecting the affinity of RNase H for RNA:DNA duplexes. In contrast to the irreversible inactivation by mutation described in the above cited U.S. Pat. No. 7,560,232, the present invention employs techniques wherein inactivation is reversed and the amino acid sequence need not be altered.

In one embodiment, these methods may involve removal and replacement of magnesium ion. Removal may be accomplished, for example, by the use of chelating agents. Such agents effectively remove magnesium ion from contact with the RNase H. Such chelating agents include, for example, ethylenediaminetetraacetic acid (EDTA), diethylene triamine pentaacetic acid (DPTA), ethylene glycol tetraacetic acid (EGTA), and 1,2-bis(o-aminophenoxy)ethane-N,N,N′,N′-tetraacetic acid (BAPTA) and many others. Any reagent capable of binding magnesium and preventing access to RNase H could be used.

Alternatively, the active site of RNase H can be replaced by other divalent metal ions, such as calcium, which ions do not permit RNase H catalytic activity. When the RNase H is to be reactivated, these ions may be removed, for example, using chelating agents and replaced by magnesium ions.

In still another approach, a reversible modification to the protein, such as formation of disulfides from cysteine residues which modification can be reversed could be used. Disulfide formation can be reversed by adding reducing agents such as dithiothreitol.

All of the foregoing methods are easily effected by altering the composition of the medium in which the RNase H is included. Thus, buffers containing chelating agents may be used effectively to inactivate the catalytic activity and this can be restored by replacing the medium, typically a buffer, with a medium that contains magnesium ions and no chelating agent.

The following examples are offered to illustrate but not to limit the invention.

### General Methods and Reagents

Buffers:

WC Buffer (Washing/Conjugation Buffer)

10×PBS pH 7.4 (Gibco® catalog number 70011-044) diluted 10 times, plus 0.1% Triton® X-100, 5 mM DTT, 1 mM EDTA.

HB Buffer (RNA/DNA Hybridization Buffer/RNase H RNA-DNA Hybrid Binding Buffer)

600 mM NaCl, 5 mM sodium phosphate, pH 7.4, 0.1% Triton® X-100, 5 mM DTT, 1 mM EDTA.

EB Buffer=Elution Buffer

10×PBS pH 7.4 (Gibco® catalog number 70011-044) plus 0.1% Triton® X-100, mM DTT, and 5 mM MgCl₂.

Coupling of Streptavidin-Coated Magnetic Beads to RNase H:SBP Fusion Protein

M-280 Dynabeads® coated with streptavidin (Invitrogen Catalog number 112-05D) were used as a magnetic solid support for immobilizing RNase H:SBP (streptavidin binding peptide) fusion protein. 12 uL beads (with 10 pmol streptavidin binding sites/uL) were washed 3 times with 100 uL WC buffer in non-stick, RNase-free 1.5 mL microtubes (Ambion part number AM12450) by gently re-suspending them in wash buffer, followed by magnetic capture and buffer elimination. Magnetic captures were done for 2 minutes using a DynaMag™-2 magnet (Invitrogen Catalog number 12321D). After the last wash, beads were re-suspended in 100 uL fresh WC buffer and 20 uL (of 10 pmol/uL) RNase H:SBP was added to the bead suspension and gently re-suspended.

The RNase H fusion protein was incubated with the beads for 30 minutes at room temperature (21° C.) while rotating at 80 rpm in a Dynal® model 10101 rotisserie mixing device. Following conjugation, beads and liquid were transferred to a new non-stick micro-centrifuge tube for washing and three, 500 uL WC buffer washes performed. The beads were re-suspended in 100 uL WC buffer and transferred to new non-stick tubes, and the beads were captured and re-suspended in 100 uL HB buffer, magnetically captured, re-suspended in 12 uL HB buffer and stored on ice.

Conjugation of Streptavidin-Coated Polystyrene Beads with the RNase H:SBP Fusion Protein

Streptavidin-coated 15 um polystyrene beads (Spherotech) were conjugated with RNase H:SBP fusion protein in a manner similar to that used to conjugate the M-280 magnetic beads Dynabeads®, except that centrifugation was used for washes instead of a magnet. The 15 um diameter polystyrene beads were used for microfluidic experiments (Example 4), where these larger beads were easier to manipulate than the smaller (2.8 um) Dynabeads®.

Capture DNA Oligonucleotides

1) 18S rRNA capture oligo (matches NCBI accession
number: NR_003278)
(SEQ ID NO: 1)
5′CGGCCGTGCGTACTTAGACATGCATGGCTTAATCTTTGAGACAAGCAT
ATGCTACTGGCAGGATCAACC3′
2) EF1a capture oligo (matches NCBI accession
number: NM_010106)
(SEQ ID NO: 2)
5′AAGCCAGCTGCTTCCATTGGTGGGTCGTTTTTGCTGTCACCAGCAACA
TTGCCTCGTCTA3′
3) 16S rRNA capture oligo for Francisella
tularensis (matches NCBI accession number:
ABXZ01000004)
(SEQ ID NO: 3)
5′GCGTTACTCACCCGTCCGCCACTCGTCAGCATCCTAAGACCTGTTACC
GTTCGACTTGCA3′
4) 16S rRNA forward RT-PCR primer for detecting
SEQ ID NO: 3:
(SEQ ID NO: 4)
5′TGCAAGTCGAACGGTAACAG3′
5) 16S rRNA reverse RT-PCR primer for detecting
SEQ ID NO: 3:
(SEQ ID NO: 5)
5′GCGTTACTCACCCGTCC3′
6) 16S rRNA probe RT-PCR sequence for detecting
SEQ ID NO: 3:
(SEQ ID NO: 6)
5′CGCCACTCGTCAGCATCCTAAGA3′

For flow cytometry assays, 5′ fluorescein amidite (FAM) fluorescent label was coupled to the EF1a capture oligo (SEQ ID NO:2). For microfluidic assays, 5′ FAM fluorescent label was coupled to the 18S capture oligo (SEQ ID NO:1). For detection of SEQ ID NO:3 by RT-PCR, a mixture of forward and reverse primers (SEQ ID NO:4 and NO:5) and a probe sequence (SEQ ID NO:6) labeled as the 5′ end with a FAM label and labeled at the 3′ end with a carboxytetramethylrhodamine (TAMRA) quencher were used.

RNA Samples

Two RNA samples were used in the Examples below:

Sample #1: Total RNA purified from mouse heart or kidney tissue using TRIzol® according to manufacturer's methods, with traces of DNA removed using RNase-free DNase.

Sample #2: In vitro-transcribed RNA, corresponding to the mouse EF1a transcript (NCBI accession NM_{—}010106, bases 890-1425)

Sample #3: Total RNA purified from Francisella tularensis subsp novacida using the Qiagen-RNeasy® Midi Kit according to manufacturer's methods.

Sample #4: Mouse lung tissue samples preserved by formalin fixation/paraffin embedding (FFPE).

Hybridization of DNA Capture Oligonucleotide to RNA Sample

One pmol of capture oligonucleotide of SEQ ID NO:1 or 2 was combined with 5 ug gamma-irradiated polyinosinic-cytidylic acid (Sigma, catalog number P0913-10 MG) in 50 uL of HB buffer in a non-stick tube on ice. Ice cold RNA sample in 2.5 uL volume and concentrations ranging from 2.5 to 10⁶ pg, diluted 10-fold serially down to 0.25 pg was added for each sample, in addition to a “no RNA” negative control. This was done in triplicate. Tubes were heated to 65° C. in an Eppendorf™ Thermomix™ for 10 minutes to remove secondary structure in the DNA or RNA, and transferred immediately to ice for >2 min. The RNA/DNA mixtures were transferred to a 22° C. Thermomix™ and allowed to hybridize for 30 minutes.

Binding RNA-DNA Duplexes to the RNaseH-Conjugated Magnetic Beads

One uL of the RNase H-conjugated magnetic beads was added to the RNA-DNA hybridization mix prepared as described above. Binding was performed in a Thermomix™ while interval mixing for 5 seconds at 1400 rpm, followed by 2 minutes resting, for a total of 30 minutes at 22° C. Following completion of the binding, the beads were captured with a magnet, re-suspended in 100 uL WC buffer, and transferred to fresh 1.5 mL non-stick tubes. The beads were washed 4 times in 500 uL WC buffer and then gently re-suspended each time after magnetic capture. Following the last 500 uL wash, the beads were magnetic captured, re-suspended in 100 uL WC buffer, transferred to a new 1.5 mL non-stick tube, magnetic captured and the WC buffer was removed.

Binding RNA-DNA Duplexes to RNase H-Conjugated Polystyrene Beads

The procedure was similar to that set forth in the previous paragraph except that the beads were trapped in a microfluidic device. See Example 4 below for details.

Elution of Capture Oligomer

Magnetic beads bound to the RNA:DNA hybrids were placed in 50 uL of EB buffer, placed in a Thermomix™ set at 22° C. for 30 minutes and interval mixed with 5 seconds at 1400 rpm alternating with 2 minutes resting. The elution solution (50 uL) containing DNA was collected after magnetic capture of the beads.

Similar procedures for elution of the DNA from polystyrene beads were performed except that the beads were trapped in a microfluidic device. See example #4 below for details.

### Example 1

### Flow Cytometry Quantification of RNA:DNA and DNA Release

In vitro-transcribed mouse EF1a RNA described above was hybridized with a 5′ FAM fluorescent labeled capture oligonucleotide of SEQ ID NO:2, treated with RNase H-conjugated magnetic beads, and analyzed using flow cytometry before and after elution. The RNA:DNA hybrid sample bound to the beads gave a fluorescence signal 4× higher (196 units) than a negative control that contained the fluorescent DNA and no RNA (47 units). After release of bound DNA:RNA hybrids from the RNase H-conjugated magnetic beads by adding elution buffer containing MgCl₂, the fluorescence of the beads decreased 4-fold to a level (45 units) that was equivalent to the background fluorescence level (44 units). An equivalent background level of bead fluorescence was observed under 3 conditions; 1) beads without added labeled DNA (44 units); 2) beads with labeled DNA, and without complementary RNA (47 units); and 3) beads after the addition of MgCl₂ in EB (45 units). These findings show the successful binding of RNase H beads for RNA:DNA hybrids, the successful release of probe DNA in the presence of Mg⁺², and the low level of non-specific binding of DNA to the RNase H-conjugated magnetic beads.

### Example 2

qRT-PCR Quantification of RNA:DNA Capture by RNase H-Conjugated Magnetic Beads

A dilution series of total RNA purified from mouse heart tissue described above (0.001 pg to 100 pg) was hybridized with 18S rRNA capture oligonucleotide SEQ ID NO:1 and treated with RNase H-conjugated magnetic beads. The beads were washed, treated with EB and the eluted DNA oligomer was analyzed by qRT-PCR, using the TaqMan fast real time PCR kit and protocol (Applied Biosystems, #4352042). The 18S ribosomal RNA capture oligonucleotide was detected with the Applied Biosystems RT-PCR assay (ID Hs03003631_g1), on an Applied Biosystems 7900 RT-PCR system.

The qRT-PCR signal was linear across the concentration range examined (R²=0.979), demonstrating quantitative RNA capture using the RNaseH method and linear performance across 5 orders of magnitude of RNA abundance.

### Example 3

### Estimation of Cross-Hybridization During Hybrid Capture

Total RNA from mouse (Sample #1) or total RNA from bacteria (Sample #3) were mixed with a capture oligo (SEQ ID NO:3) specific for bacterial 16S RNA in 50 μl reactions as described above (Hybridization), except that no DTT was included in the hybridization buffer. The bacterial 16S RNA capture probe is predicted to hybridize with 16S RNA along its entire length of 60 nucleotides. By comparison, the capture probe is predicted to exhibit undesired cross-hybridization to short regions of sequence complementary (of up to 14 nucleotides) within the mouse RNA sample. Two different amounts of RNA were used for each: 2.5 ng and 25 ng, as well as a negative control containing no RNA. Samples were hybridized at 65° C. for 1 hr. 5 units of a mixture of RNase A and RNase T1 (Fermentas catalog #EN0551) were then added to each sample. The samples were incubated an additional 10 minutes at 65° C., and were then incubated at 52° C. for 5 minutes. At this point, 0.25 μl of 1M DTT was added to each sample. Binding of the hybrids to the beads was performed as described above, except that the incubation occurred at 52° C. for 1 hr. Washing and elution of the sample was performed as described above. The eluate from each sample was assayed for the presence of the capture oligo (SEQ ID NO:3) using RT-PCR with SEQ ID NO:4, SEQ ID NO:5, and SEQ ID NO:6 as the detection primers and probe.

RT-PCR results for the samples containing mouse RNA incubated with the bacterial capture probe revealed no significant increase (5.7×10³ units for 2.5 ng and 5.0×10³ units for 25 ng) above the no-RNA control (5.2×10³ units). By comparison, RT-PCR results for samples containing bacterial RNA exhibited a significant increase over background with linear scaling (4.0×10⁵ units for 2.5 ng and 3.2×10⁶ units for 25 ng). This demonstrates that the method is resistant to cross-hybridization of the DNA capture oligo to RNA sequences with short regions of sequence complementarity to the capture oligo (up to 14 base pairs).

### Example 4

### Micro-Scale Profiling of RNA

RNA was captured from nanoliter-scale volume samples to approximate capture and detection of the amount of RNA present in a single cell. A poly-dimethylsiloxane (PDMS) microfluidic device was constructed using standard methods, for example, as described in Unger, M. A., et al., Science (2000) 288:113-116. The device has 4 reagent inputs, which were loaded with:

(A) HB buffer+40 mM DTT,

(B) 15 μm-diameter polystyrene beads in phosphate buffered saline solution+21% glycerol,

(C) 1 μm RNase H in HB buffer+40 mM DTT,

(D) EB buffer containing 5 mM MgCl2+40 mM DTT.

The device also contains 1 sample input. This input was loaded with a mixture of 1 μg of purified mouse total RNA and 1 pmol of 18S rRNA capture oligonucleotide SEQ ID NO:1 with a 5′ FAM fluorescent label, hybridized (as described above) in HB buffer. All reagent inputs were pressurized at 1.5 psi above atmosphere. The reagent multiplexed input and sample input are connected to a ring-shaped reactor that contains a sieve valve for trapping 15 μm beads as well as additional valves to enable peristaltic pumping and mixing within the reactor. Finally, the reactor has outlet ports for removal of the waste stream and for elution of DNA oligo samples to be analyzed.

The beads were trapped behind a sieve valve in a sample chamber, and washed for 1 minute with HB buffer. RNase H was loaded onto the beads and mixed using 1 Hz peristaltic pumping for 20 minutes, allowing binding to the beads. The beads were again washed for 1 minute with HB buffer. 15 nL of the hybridized sample was then introduced into the reactor and mixed with the trapped beads using 1 Hz peristaltic pumping for 1 hr. The bead column was washed for 2 minutes with HB buffer. HB buffer was flowed through the column and collected at the elution port for 1 minute (as a negative control). EB buffer was introduced to the bead column for 15 seconds and collected at the elution port (experimental sample). Liquid flow was stopped, and hydrolysis was allowed to proceed for 1 minute. The column was then flushed with EB buffer for an additional 45 seconds and collected at the elution port (experimental sample). Both samples (approximately 2 ul in volume) were diluted with 20 ul of distilled water and analyzed using qRT-PCR directed at the 18S oligo as described above.

Signal from the elution sample contained 3.3 times more DNA than the negative control sample, demonstrating that the hybrid capture protocol can be successfully implemented on a microfluidic device.

### Example 5

### Results with FFPE Samples

Mouse lung samples were obtained as FFPE sections mounted on a microscope slide (BioChain cat #T2334152) and were dewaxed using EZ-Dewax™ (BioGenex cat #HK585-5K) according to the manufacturer's instructions. RNA was extracted from 2 slides using the Qiagen RNeasy® FFPE kit according to manufacturer's instructions.

The resulting RNA was pure as judged by UV absorbance at 260 nm and 280 nm, but was highly degraded as judged by electrophoresis (on an Agilent Bioanalyzer 2100). 1 ng of the degraded RNA was treated using the method of the invention described above. For comparison, identical samples were used for a series of cDNA synthesis reactions on 18s RNA.

For cDNA synthesis, reverse transcription primer sequences were chosen within the 18s RNA sequence at various distances from the qPCR amplicon used to detect 18s RNA. The reverse transcription primer located nearest to the qPCR amplicon is expected to have the least chance of the process of cDNA synthesis being interrupted by damaged RNA. As the RT primer sequence is moved more distant to the qPCR amplicon, the chance of being interrupted is greater, so that the qPCR signal will be reduced. All samples were assayed using Taqman qPCR for 18s RNA (as described above).

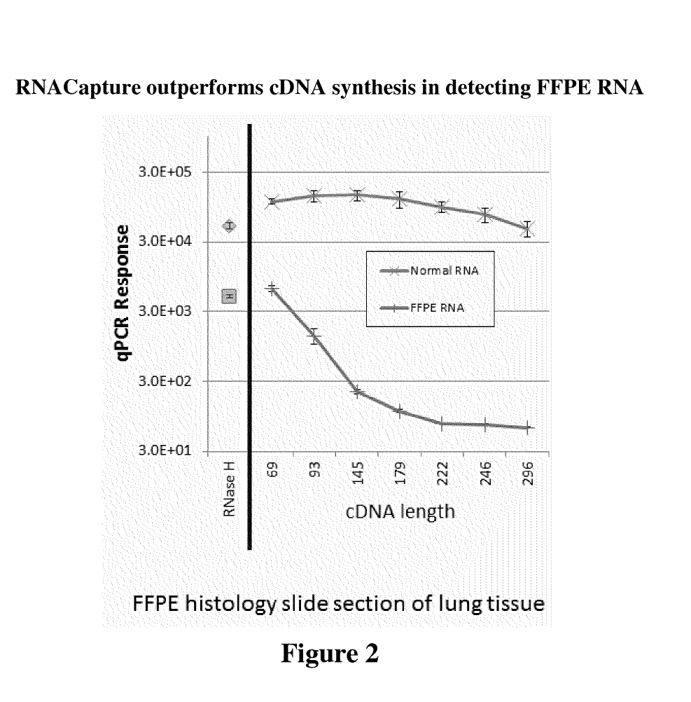

The results of the assay are shown in FIG. 2. The data on the left side of the figure represents results of the current method, and the data on the right side of the figure represents the results produced with cDNA synthesis. For cDNA synthesis, the “cDNA length” labels on the X-axis indicate the distance between the beginning of the reverse transcription primer and the end of the qPCR amplicon. On RNA derived from FFPE tissues (cDNA data points shown as ‘+’, invention method shown as a square), the RNase-H capture invention method gave results similar to those shown for cDNA synthesis when the RT primer was within the qPCR amplicon (cDNA length 69 nucleotides), but results of the cDNA synthesis method were significantly reduced as the RT primer was moved farther from the qPCR amplicon (cDNA length >69 nucleotides). For comparison, the results of a similar experiment using cDNA synthesis on high quality mouse lung RNA are shown as “intact RNA” (cDNA data points shown as ‘X’, invention method shown as a diamond).

1. A method to detect and/or quantify and/or identify at least one RNA in a sample, comprising the steps of:
a) exposing the sample to at least one DNA probe comprising a sequence complementary to said at least one RNA of interest to form DNA:RNA hybrids with any said RNA in the sample;
b) incubating the DNA:RNA hybrids formed in a) with RNase H under conditions that inhibit the nuclease activity of the RNase H but do not inhibit its DNA:RNA binding activity;
c) separating RNase H-bound DNA:RNA from unbound DNA and RNA;
d) incubating the RNase H-bound DNA:RNA under conditions wherein the nuclease activity is restored so as to hydrolyze the RNA of the DNA:RNA hybrids and release the DNA probe; and
e) determining the presence and/or amount and/or identity of the released DNA probe, thereby determining the presence and/or quantity and/or identity of the at least one RNA of interest in the sample.

2. The method of claim 1 wherein multiple RNA species of interest are detected and/or quantified and/or identified and wherein step a) employs DNA probes complementary to each said RNA of interest and wherein in step e) the presence and/or quantity and/or identity of DNA probe complementary to each of said multiple RNA's of interest is determined.

3. The method of claim 1 wherein said DNA probe comprises a sequence extension that comprises one or more labels.

4. The method of claim 2 wherein each said DNA probe comprises a sequence extension that comprises one or more labels that are different with respect to each RNA to be detected.

5. The method of claim 1 wherein the DNA probe further comprises a nucleotide bar code or one or more primer sequences.

6. The method of claim 1 wherein the released DNA probe is detected and/or quantified and/or identified by RT-PCR, sequencing, microarray, or a detectable label.

7. The method of claim 2 wherein the released DNA probes are detected and/or quantified and/or identified by RT-PCR, sequencing, microarray, or a detectable label.

8. The method of claim 1 wherein the RNase H in step b) and/or step c) is immobilized.

9. The method of claim 8 wherein said immobilization is on magnetic or non-magnetic beads.

10. The method of claim 1 which is conducted in a microfluidic system.

11. The method of claim 1 which further includes removing RNA molecules that are not of interest and/or single-stranded DNA from said sample.

12. The method of claim 11 in which the sample is treated with at least one nuclease specific for single-stranded DNA or single-stranded RNA or at least one protein that binds single-stranded RNA and/or DNA during or between steps a), b) and c).

13. The method of claim 1 wherein the conditions in step b) comprise inclusion of a chelating agent in medium in which the DNA:RNA hybrids and RNase H are contained.

14. The method of claim 1 wherein the conditions in step d) include supplying magnesium ion to a medium in which the RNase H bound DNA:RNA is contained.

15. The method of claim 1 wherein the RNase H is sufficiently thermostable to permit steps b) and/or c) and/or d) to be performed at temperature >50° C.

16. The method of claim 1 wherein the RNase H is sufficiently cold adapted to permit steps b) and/or c) and/or d) to be performed at temperatures between 1° C. and 4° C.

17. A kit for performing the method of claim 1 which comprises at least one DNA probe, RNase H, and suitable buffers.

18. A kit for performing the method of claim 1, comprising at least two of the elements selected from the group consisting of a DNA probe, an RNase H and non-magnesium containing buffers.

19. The kit of claim 18 which comprises three of said elements.

20. The kit of claim 18 which further comprises a single-stranded nucleic acid nuclease or a single-stranded DNA- or RNA-binding protein.

21. The method of claim 1 wherein the sample is a degraded RNA-containing sample.

22. The method of claim 1 wherein the sample is an FFPE sample.

23. The method of claim 1 which employs two DNA probes at adjacent positions on the RNA in said sample and a DNA ligase that operates on double-stranded nucleic acids to ligate the bound probes.
